(19) United States
(12) Patent Application Publication
(10) Pub. No.: US 2017/0228879 A1
(43) Pub. Date: Aug. 10, 2017

(54) IMAGE PROCESSING APPARATUS, CAPSULE ENDOSCOPE SYSTEM, AND ENDOSCOPE SYSTEM

(71) Applicant: OLYMPUS CORPORATION, Tokyo (JP)

(72) Inventor: Daisuke SATO, Tokyo (JP)

(73) Assignee: OLYMPUS CORPORATION, Tokyo (JP)

(21) Appl. No.: 15/498,845

(22) Filed: Apr. 27, 2017

Related U.S. Application Data
(63) Continuation of application No. PCT/JP2016/054621, filed on Feb. 17, 2016.

(30) Foreign Application Priority Data
Jun. 30, 2015 (JP) 2015-131904

(51) Int. Cl. G06T 7/521; G02B 23/24; G01S 17/02; A61B 1/04; A61B 1/00

(52) U.S. Cl. CPC G06T 7/521; A61B 1/041; A61B 1/00009; A61B 1/00045; G01S 17/023; G02B 23/2484; G06T 2207/10068; G06T 2200/24; G06T 2207/10028

(57) ABSTRACT

An image processing apparatus performs image processing based on image data and ranging data output from an image sensor. The ranging data represents a distance between the image sensor and a subject. The image sensor is configured to receive reflected light of illumination light reflected from the subject and to output the image data and the ranging data. The image processing apparatus includes a processor configured to: calculate a parameter of the illumination light emitted onto a point on the subject, based on the ranging data; calculate a parameter of the reflected light, based on a gradient of a depth on the point on the subject calculated from the ranging data; and calculate the distance between the image sensor and the subject in a direction orthogonal to a light-receiving surface of the image sensor, based on the image data and the parameters of the illumination light and the reflected light.

### CROSS REFERENCES TO RELATED APPLICATIONS

This application is a continuation of PCT international application Ser. No. PCT/JP2016/054621, filed on Feb. 17, 2016 which designates the United States, incorporated herein by reference, and which claims the benefit of priority from Japanese Patent Application No. 2015-131904, filed on Jun. 30, 2015, incorporated herein by reference.

### BACKGROUND

1. Technical Field

The disclosure relates to an image processing apparatus for performing image processing based on data obtained by imaging an inside of a living body. The disclosure also relates to a capsule endoscope system and an endoscope system.

2. Related Art

Endoscope systems have been widely used to perform diagnosis of the living body by introducing an endoscope into a living body to observe images of a subject captured by the endoscope. In recent years, endoscope systems incorporating a ranging system for measuring a distance (depth) from the endoscope to the subject have been developed.

As an exemplary ranging system, JP 2013-232751 A discloses a system that includes, in an imaging unit, an image sensor for image plane phase difference detection auto-focus (AF), and that measures the depth to the subject on the basis of an output signal from a ranging pixel disposed on the image sensor.

Moreover, JP 2009-267436 A discloses a system that includes, in an imaging unit, a time of flight (TOF)-system ranging sensor independently of the image sensor for generating images of the subject, and that measures the depth to the subject on the basis of the output signal from the ranging sensor.

Furthermore, JP 2009-41929 A discloses a technique of calculating the depth from the image of the subject on the basis of the positional relationship between the illumination unit that illuminates the subject, and the imaging unit. Specifically, the depth to the subject is calculated using an emission angle (angle with respect to the optical axis of the illumination unit) of the light emitted from the illumination unit and incident on a point of interest on the subject, and using an imaging angle (angle with respect to the optical axis of collection optical system) of the light that is reflected from the point of interest and incident on the imaging unit via the collection optical system.

### SUMMARY

In some embodiments, an image processing apparatus performs image processing based on image data and ranging data output from an image sensor. The ranging data represents a distance between the image sensor and a subject. The image sensor is configured to receive reflected light of illumination light reflected from the subject and to output the image data and the ranging data. The image processing apparatus includes a processor having hardware. The processor is configured to: calculate a parameter of the illumination light emitted onto a point on the subject, based on the ranging data; calculate a parameter of the reflected light, based on a gradient of a depth on the point on the subject calculated from the ranging data; and calculate the distance between the image sensor and the subject in a direction orthogonal to a light-receiving surface of the image sensor, based on the image data, the parameter of the illumination light, and the parameter of the reflected light.

In some embodiments, a capsule endoscope system includes the image processing apparatus and a capsule endoscope configured to be introduced into the subject.

In some embodiments, an endoscope system includes the image processing apparatus and an endoscope configured to be inserted into the subject.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating an exemplary configuration of a ranging system according to a first embodiment of the present invention;

FIG. 2 is a schematic diagram illustrating a light-receiving surface of an image sensor illustrated in FIG. 1;

FIG. 3 is a block diagram illustrating a configuration of a depth calculation unit illustrated in FIG. 1;

FIG. 4 is a schematic diagram for illustrating a principle of measuring a subject distance;

FIG. 5 is a schematic diagram for illustrating distribution characteristics of illumination light;

FIG. 6 is a schematic diagram for illustrating an image region of a depth image corresponding to the light-receiving surface of the image sensor illustrated in FIG. 4;

FIG. 7 is a schematic diagram for illustrating a method for calculating a depth gradient;

FIG. 8 is a schematic diagram for illustrating a method for calculating a depth gradient;

FIG. 9 is a schematic diagram for illustrating distribution characteristics of reflected light;

FIG. 10 is a schematic diagram illustrating an exemplary configuration of a ranging system according to a second embodiment of the present invention;

FIG. 11 is a schematic diagram for illustrating an exemplary screen displayed on a display unit illustrated in FIG. 10;

FIG. 12 is a schematic diagram for illustrating a principle of measuring a distance on a subject, corresponding to a distance between two points within the image;

FIG. 13 is a schematic diagram for illustrating a principle of measuring a distance on a subject, corresponding to a distance between two points within the image;

FIG. 14 is a schematic diagram illustrating an exemplary configuration of an endoscope system according to a third embodiment of the present invention;

FIG. 15 is a schematic diagram illustrating an exemplary internal structure of a capsule endoscope illustrated in FIG. 14; and

FIG. 16 is a schematic diagram illustrating an exemplary configuration of an endoscope system according to a fourth embodiment of the present invention.

### DETAILED DESCRIPTION

Hereinafter, an image processing apparatus, a ranging system, and an endoscope system according to embodiments of the present invention will be described with reference to the drawings. The drawings merely schematically illustrate shapes, sizes, and positional relations to the extent that contents of the present invention are understandable. Accordingly, the present invention is not limited only to the shapes, sizes, and positional relations exemplified in the drawings. The same reference signs are used to designate the same elements throughout the drawings.

### First Embodiment

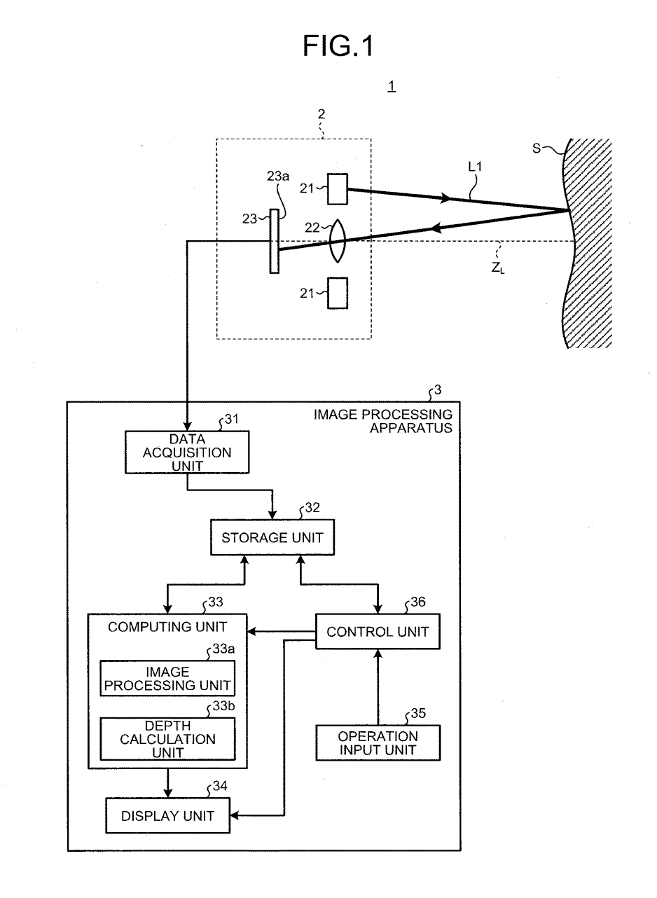

FIG. 1 is a schematic diagram illustrating an exemplary configuration of a ranging system according to a first embodiment of the present invention. A ranging system 1 according to the first embodiment is a system that is applied to an endoscope system, or the like, that is introduced into a living body to perform imaging and that measures the distance (depth) to a subject such as mucosa. The endoscope system may be a typical endoscope system having a video scope including an imaging unit at a distal end portion of an insertion unit inserted into the subject, or alternatively, may be a capsule endoscope system including a capsule endoscope configured to be introduced into the living body. The capsule endoscope includes an imaging unit and a wireless communication unit in a capsule-shaped casing, and is configured to perform image capturing.

As illustrated in FIG. 1, the ranging system 1 includes an imaging unit 2 that images a subject S, thereby generating and outputting image data, and actually measures the distance to the subject S, thereby generating and outputting ranging data, and includes an image processing apparatus 3 that obtains the image data and the ranging data output from the imaging unit 2 and creates an image of the subject S on the basis of the image data, as well as creates a depth map to the subject S using the image data and the ranging data.

The imaging unit 2 includes an illumination unit 21 configured to emit illumination light to irradiate a subject S, a collection optical system 22 such as a condenser lens, and an image sensor 23.

The illumination unit 21 includes a light emitting element such as a light emitting diode (LED), and a drive circuit for driving the light emitting element. The illumination unit 21 generates white light or illumination light with a specific frequency band and emits the light onto the subject S.

The image sensor 23 is a sensor capable of obtaining image data representing visual information on the subject S and ranging data representing the depth to the subject S. The image sensor 23 includes a light-receiving surface 23a that receives illumination light (namely, reflected light) emitted from the illumination unit 21, reflected from subject S, and collected by the collection optical system 22. In the first embodiment, an image plane phase difference detection AF sensor is employed as the image sensor 23.

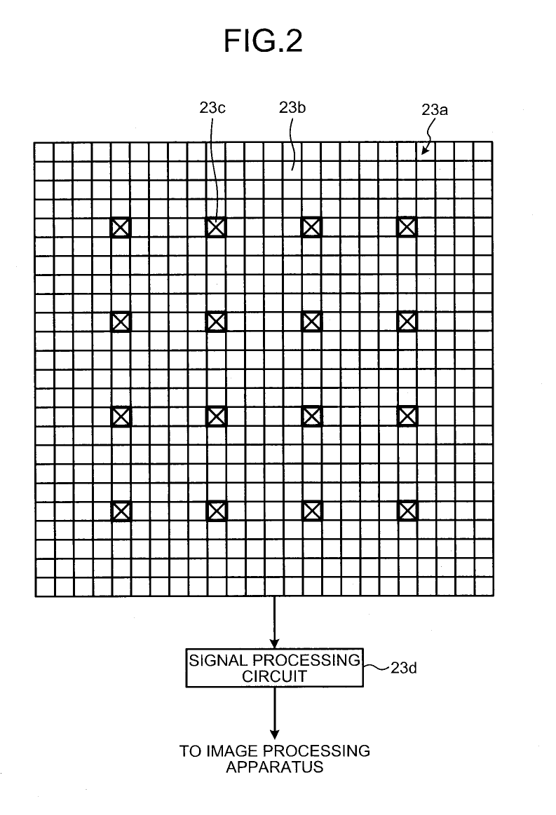

FIG. 2 is a schematic diagram for illustrating a configuration of the image sensor 23. As illustrated in FIG. 2, the image sensor 23 includes a plurality of imaging pixels 23b, ranging pixels 23c, and a signal processing circuit 23d. The plurality of imaging pixels is arranged on the light-receiving surface 23a. The signal processing circuit 23d processes an electrical signal output from these pixels. A plurality of imaging pixels 23b is arrayed in a matrix on the light-receiving surface 23a. A plurality of ranging pixels 23c is arranged so as to replace a portion of the matrix of the plurality of imaging pixels 23b. In FIG. 2, check marks are drawn for the positions of the ranging pixels 23c to be distinguished from the imaging pixels 23b.

Each of the imaging pixels 23b has a structure including a microlens and any of color filters red (R), green (G), and blue (B) stacked on a photoelectric conversion unit such as a photodiode, and generates an electric charge corresponding to the amount of light incident onto the photoelectric conversion unit. The imaging pixels 23b are arranged in a predetermined order, such as a Bayer array, in accordance with the color of the color filter included in each of the pixels. The signal processing circuit 23d converts the electric charge generated by each of the imaging pixels 23b into a voltage signal, and further converts the voltage signal into a digital signal, thereby outputting the signal as image data.

Each of the ranging pixels 23c has a structure in which two photoelectric conversion units are arranged on a same plane side by side and furthermore, one microlens is disposed so as to be placed across these photoelectric conversion units. The light incident onto the microlens is further incident onto the two photoelectric conversion units at the distribution ratio corresponding to the incident position at the microlens. Each of the two photoelectric conversion units generates an electric charge corresponding to the amount of incident light. The signal processing circuit 23d converts the electric charge generated at the two photoelectric conversion units of the ranging pixel 23c into a voltage signal, generates and outputs ranging data representing a distance (depth) from the imaging unit 2 to the subject S on the basis of the phase difference (information regarding distance) between these voltage signals.

The image processing apparatus 3 includes a data acquisition unit 31 that obtains image data and ranging data output from the imaging unit 2, a storage unit 32 that stores the image data and the ranging data obtained by the data acquisition unit 31 and stores various programs and parameters used on the image processing apparatus 3, a computing unit 33 that performs various types of calculation processing on the basis of the image data and the ranging data, a display unit 34 that displays an image of the subject S, or the like, an operation input unit 35 that functions as an input device for inputting various types of information and commands into the image processing apparatus 3, and a control unit 36 for performing overall control of these elements.

The data acquisition unit 31 is appropriately configured in accordance with a mode of the endoscope system to which the ranging system 1 is applied. For example, in the case of a typical endoscope system configured to insert a video scope into the body, the data acquisition unit 31 includes an interface that captures the image data and the ranging data generated by the imaging unit 2 provided at the video scope. Moreover, in the case of a capsule endoscope system, the data acquisition unit 31 includes a receiving unit that receives a signal wirelessly transmitted from a capsule endoscope via an antenna. Alternatively, image data and ranging data may be sent and received to and from the capsule endoscope, using a portable storage medium. In this case, the data acquisition unit 31 includes a reader apparatus that removably attaches the portable storage medium and reads out the stored image data and ranging data. Alternatively, in a case where a server to store the image data and the ranging data generated in the endoscope system is installed, the data acquisition unit 31 includes a communication apparatus, or the like, to be connected with the server, and obtains various types of data by performing data communication with the server.

The storage unit 32 includes an information storage apparatus including various types of integrated circuit (IC) memories such as renewable flash memories including a read only memory (ROM) and a random access memory (RAM), a hard disk that is either internal or connected with a data communication terminal, or a compact disc read only memory (CD-ROM), together with an apparatus for reading information from and writing information into the information storage apparatus. The storage unit 32 stores programs for operating the image processing apparatus 3 and causing the image processing apparatus 3 to execute various functions, data used in execution of the program, specifically, image data and ranging data obtained by the data acquisition unit 31, and various parameters.

The computing unit 33 includes a general-purpose processor such as a central processing unit (CPU), a dedicated processor including various calculation circuits such as an application specific integrated circuit (ASIC), for executing specific functions, or the like. In a case where the computing unit 33 is a general-purpose processor, it executes calculation processing by reading various calculation programs stored in the storage unit 32. In another case where the computing unit 33 is a dedicated processor, the processor may execute various types of calculation processing independently, or the processor may execute calculation processing in cooperation with or combined with the storage unit 32 using various data stored in the storage unit 32.

Specifically, the computing unit 33 includes an image processing unit 33a that performs predetermined image processing such as white balance processing, demosaicing, gamma conversion, smoothing (noise removal, etc.) on the image data, thereby generating image for display, and includes a depth calculation unit 33b that calculates a depth (distance from the collection optical system 22) to the subject S corresponding to each of the pixel positions within the image for display, created by the image processing unit 33a, on the basis of the image data and the ranging data The configuration and operation of the depth calculation unit 33b will be described in detail below.

The display unit 34 includes various types of displays formed of liquid crystal, organic electroluminescence (EL), or the like, and displays an image for display created by the image processing unit 33a, and information such as the distance calculated by the depth calculation unit 33b, or others.

The control unit 36 includes a general-purpose processor such as a CPU, and a dedicated processor including various calculation circuits such as an ASIC, for executing specific functions. In a case where the control unit 36 is a general-purpose processor, the processor performs overall control of the image processing apparatus 3, including transmission of an instruction and data to each element of the image processing apparatus 3, by reading a control program stored in the storage unit 32. In another case where the control unit 36 is a dedicated processor, the processor may execute various types of processing independently, or the processor may execute various types of processing in cooperation with or combined with the storage unit 32 using various types of data stored in the storage unit 32.

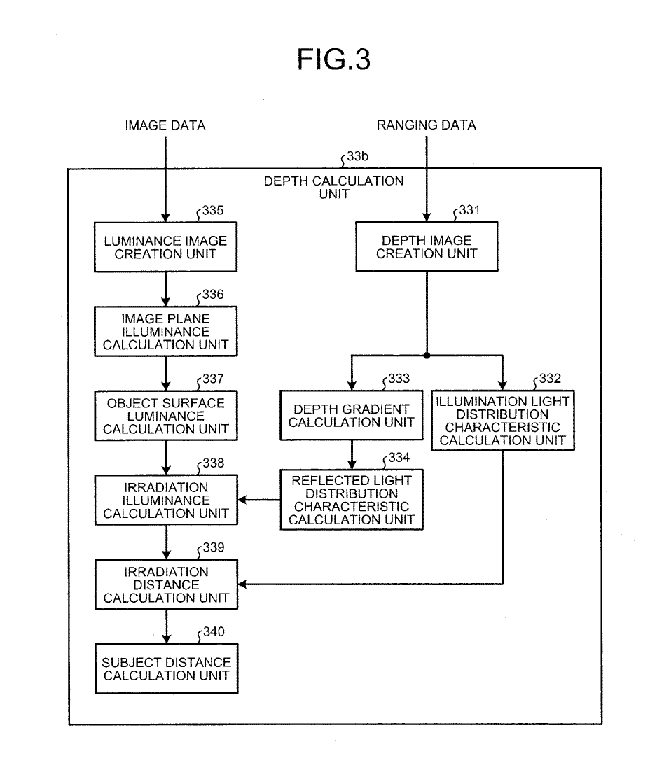

FIG. 3 is a block diagram illustrating a detailed configuration of the depth calculation unit 33b. As illustrated in FIG. 3, the depth calculation unit 33b includes a depth image creation unit 331, an illumination light distribution characteristic calculation unit 332, a depth gradient calculation unit 333, a reflected light distribution characteristic calculation unit 334, a luminance image creation unit 335, an image plane illuminance calculation unit 336, an object surface luminance calculation unit 337, an irradiation illuminance calculation unit 338, an irradiation distance calculation unit 339, and a subject distance calculation unit 340.

The depth image creation unit 331 creates a depth image in which the depth between a point on the subject S corresponding to each of the pixel positions within an image for display created by the image processing unit 33a and the collection optical system 22 is defined as a pixel value of each of the pixels, on the basis of the ranging data read from the storage unit 32. As described above, since the ranging pixels 23c are sparsely arranged on the light-receiving surface 23a, the depth image creation unit 331 calculates the depth for the pixel position on which the ranging pixel 23c is not disposed, by interpolation calculation using the ranging data output from the ranging pixel 23c disposed in vicinity.

The illumination light distribution characteristic calculation unit 332 calculates a value in a radiation angle direction on the light distribution characteristic, as a parameter of the illumination light emitted onto the subject S, on the basis of the depth image created by the depth image creation unit 331.

The depth gradient calculation unit 333 calculates a gradient of depth (depth gradient) on a point of the subject S on the basis of the depth image created by the depth image creation unit 331.

The reflected light distribution characteristic calculation unit 334 calculates a value in a reflection angle direction on the light distribution characteristic, as a parameter of the illumination light reflected from the subject S (that is, reflected light), on the basis of the depth gradient calculated by the depth gradient calculation unit 333.

The luminance image creation unit 335 creates a luminance image defining the luminance of the image of the subject S as a pixel value of each of the pixels on the basis of the image data read from the storage unit 32.

The image plane illuminance calculation unit 336 calculates illuminance on the image plane of the image sensor 23 on the basis of the luminance image created by the luminance image creation unit 335.

The object surface luminance calculation unit 337 calculates the luminance on the surface of the subject S on the basis of the illuminance on the image plane calculated by the image plane illuminance calculation unit 336.

The irradiation illuminance calculation unit 338 calculates irradiation illuminance of the illumination light emitted onto the subject S, on the basis of the luminance of the object surface calculated by the object surface luminance calculation unit 337 and on the basis of the value in the reflection angle direction of the distribution characteristics of the reflected light calculated by the reflected light distribution characteristic calculation unit 334.

The irradiation distance calculation unit 339 calculates an irradiation distance from the collection optical system 22 to the subject S on the basis of the irradiation illuminance of the illumination light emitted onto the subject S and on the basis of the value in the radiation angle direction of the distribution characteristics of the illumination light calculated by the illumination light distribution characteristic calculation unit 332.

The subject distance calculation unit 340 calculates a subject distance, that is, an irradiation distance calculated by the irradiation distance calculation unit 339, projected onto an optical axis Z_{L} of the collection optical system 22.

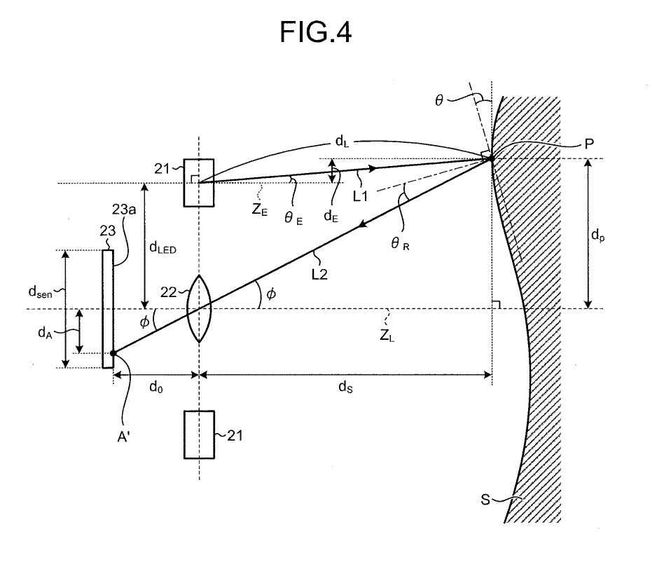

Next, a ranging method according to the first embodiment will be described in detail with reference to FIGS. 1 to 8. FIG. 4 is schematic diagram for illustrating positional and angular relationships between the subject S and each element of the imaging unit 2.

First, the ranging system 1 emits illumination light L1 onto the subject S by causing the illumination unit 21 to emit light. With this process, the illumination light reflected from the subject S (that is, reflected light) is collected by the collection optical system 22 and becomes incidence on the light-receiving surface 23a of the image sensor 23. On the basis of an electric signal output individually from the imaging pixel 23b and the ranging pixel 23c arranged on the light-receiving surface 23a, the signal processing circuit 23d (refer to FIG. 2) outputs image data for the position on each of the imaging pixels 23b, as well as outputs ranging data for the position of each of the ranging pixels 23c. The data acquisition unit 31 of the image processing apparatus 3 captures the image data and the ranging data and stores the data in the storage unit 32.

As illustrated in FIG. 3, the depth calculation unit 33b captures the ranging data and the image data from the storage unit 32, then, inputs the ranging data into the depth image creation unit 331, while inputting the image data into the luminance image creation unit 335.

The depth image creation unit 331 creates a depth image of a size corresponding to the entire light-receiving surface 23a by defining a distance d_{S} (refer to FIG. 4) from the collection optical system 22 to the subject S as a pixel value of each of the pixels on the basis of the input ranging data. As illustrated in FIG. 2, the ranging pixels 23c are sparsely arranged on the light-receiving surface 23a of the image sensor 23. Accordingly, the depth image creation unit 331 calculates a value using ranging data based on an output value from the ranging pixel 23c for the pixel within the depth image that is corresponding to the position of the ranging pixel 23c, and calculates a value by interpolation using ranging data for other pixels within the depth image. Accordingly, the distance d_{S} on the depth image, on the pixel position for which a measurement value based on the output value from the ranging pixel 23c has not been obtained, is an approximate value that has not received any feedback on irregularities of the surface of the subject S, or the like.

Subsequently, on the basis of the depth image created by the depth image creation unit 331, the illumination light distribution characteristic calculation unit 332 calculates a value in the radiation angle direction of the distribution characteristics of the illumination light L1 emitted onto each of the points (e.g. a point of interest P) on the subject S.

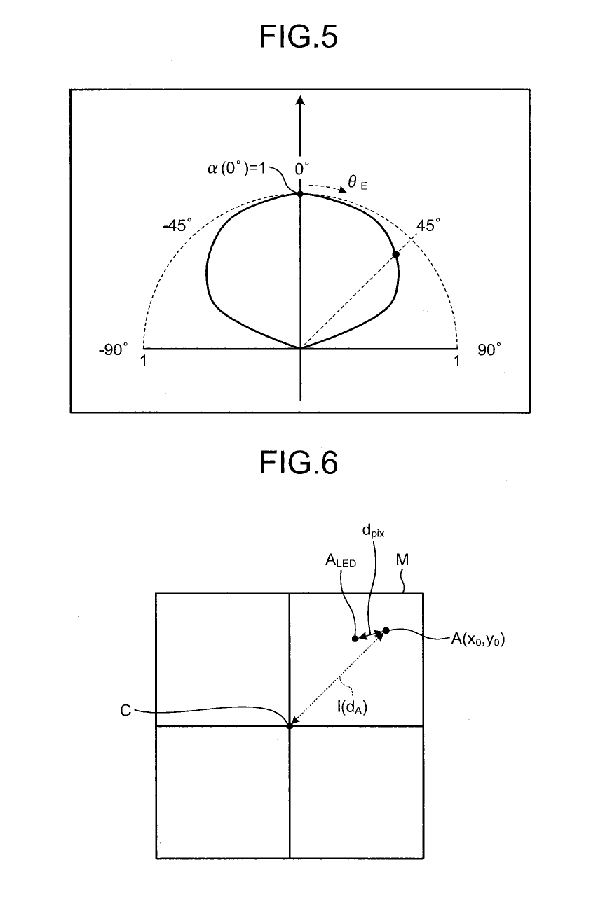

FIG. 5 illustrates a relationship between a radiation angle θ_{E} formed by the radiation direction of the illumination light L1 with respect to an optical axis Z_{E} of the illumination unit 21, and a value α(θ_{E}) in the radiation angle direction on the light distribution characteristic corresponding to the radiation angle θ_{E}. In FIG. 5, normalization is performed on the basis of the maximum light intensity on a radiation surface, that is, the light intensity when the radiation angle θ_{E}=0°. The illumination light distribution characteristic calculation unit 332 reads, from the storage unit 32, a function or a table indicating light distribution characteristic illustrated in FIG. 5, calculates the radiation angle θ_{E} from the positional relationship between the illumination unit 21 and the point of interest P, and calculates the value α(θ_{E}) in the radiation angle direction on the light distribution characteristic corresponding to the radiation angle θ_{E}.

A typical LED has light distribution characteristics represented in cosine, and thus, in a case where the radiation angle θ_{E}=45°, light intensity α(45°) in the radiation angle direction is obtained by multiplying the value α(0°) at the radiation angle θ_{E}=0° by cos(45°).

Now a method for calculating the radiation angle θ_{E} will be described. FIG. 6 is a schematic diagram illustrating an image region of a depth image corresponding to the light-receiving surface 23a of the image sensor 23. First, the illumination light distribution characteristic calculation unit 332 extracts a pixel A′ (refer to FIG. 4) on the light-receiving surface 23a, that corresponds to a pixel of interest A (x₀, y₀) within a depth image M, and converts the coordinate value of the pixel A′ from pixel into distance (mm) using the number of pixels of the image sensor 23 and a sensor size dₛₑₙ (mm). Moreover, the illumination light distribution characteristic calculation unit 332 calculates a distance from the optical axis Z_{L} of the collection optical system 22 to the pixel A′, namely, an image height d_{A} using the coordinate value of the pixel A′ that has been converted into the distance. Then, a field angle φ is calculated by the following formula (1) on the basis of a distance (d₀) (design value) from the collection optical system 22 to the light-receiving surface 23a, and the image height d_{A}.

φ=tan⁻¹ (d_{A}/d₀) (1)

A length l(d_{A}) within the depth image M, corresponding to the image height d_{A}, is indicated in a broken line in FIG. 6.

Using the following formula (2), the illumination light distribution characteristic calculation unit 332 calculates a distance between the point of interest P on the subject S corresponding to the pixel of interest A, and the optical axis Z_{L}, namely, a height d_{P} of the subject, on the basis of the field angle φ, the pixel value of the pixel of interest A on the depth image M, namely, a depth d_{S}.

d_{P}=d_{S} tan φ (2)

Subsequently, the illumination light distribution characteristic calculation unit 332 calculates the coordinate within the depth image M, corresponding to the position of the light emitting element included in the illumination unit 21. On the imaging unit 2, a distance d_{LED} between the optical axis Z_{E} of the light emitting element included in the illumination unit 21, and the optical axis Z_{L} of the collection optical system 22, is determined as a design value; and also the positional relationship between the light emitting element and the light-receiving surface 23a of the image sensor 23 is determined as a design value. Accordingly, the illumination light distribution characteristic calculation unit 332 obtains an image height of the depth image M using the number of pixels and the sensor size dₛₑₙ(mm) of the image sensor 23, and calculates a coordinate A_{LED} of the pixel within the depth image M, corresponding to the position of the light emitting element included in the illumination unit 21 on the basis of the obtained image height.

Subsequently, the illumination light distribution characteristic calculation unit 332 calculates an interval Dₚᵢₓ of these pixels on the basis of the coordinate of the pixel of interest A and from the coordinate of a pixel A_{LED} corresponding to the position of the light emitting element. Then, the interval dₚᵢₓ is converted into the distance (mm) on the subject S using the number of pixels and the sensor size dₛₑₙ(mm) of the image sensor 23. This distance is a distance d_{E} from the point of interest P to the optical axis Z_{E} of the light emitting element. Using the following formula (3), the illumination light distribution characteristic calculation unit 332 calculates the radiation angle θ_{E} on the basis of the distance d_{E} and the depth d_{S} of the point of interest P.

θ_{E}=tan⁻¹(d_{E}/d_{S}) (3)

On the basis of the radiation angle θ_{E} calculated in this manner, the illumination light distribution characteristic calculation unit 332 calculates a value α(θ_{E}) (FIG. 5) in the radiation angle direction of the distribution characteristics of the illumination light L1.

If the illumination unit 21 has a plurality of light emitting elements, the illumination light distribution characteristic calculation unit 332 may calculate, for each of the plurality of light emitting elements, the radiation angle θ_{E} using the above-described technique, and calculate a value of the light distribution characteristics in the radiation angle direction based on the calculated plurality of radiation angles θ_{E}. In this case, a function or a table representing characteristics corresponding to the arrangement of the plurality of light emitting elements is read from the storage unit 32 to the illumination light distribution characteristic calculation unit 332. For example, in a case where the illumination unit 21 includes four light emitting elements and corresponding radiation angles θ_{E1}, θ_{E2}, θ_{E3}, and θ_{E4} of the light emitting elements are calculated for a certain point of interest P, also values α(θ_{E1}, θ_{E2}, θ_{E3}, θ_{E4}) in the radiation angle direction on the light distribution characteristics based on these radiation angles are calculated.

Referring back to FIG. 3, the depth gradient calculation unit 333 calculates a depth gradient on a point on the subject S on the basis of the depth image M (refer to FIG. 6) created by the depth image creation unit 331. The depth gradient is calculated by taking the derivative of the pixel value (namely, depth) of each of the pixels within the depth image. As illustrated in FIG. 4, the depth gradient gives a gradient (gradient angle θ) of a contact surface on the point of interest P with respect to the surface orthogonal to the optical axis Z_{L} of the collection optical system 22.

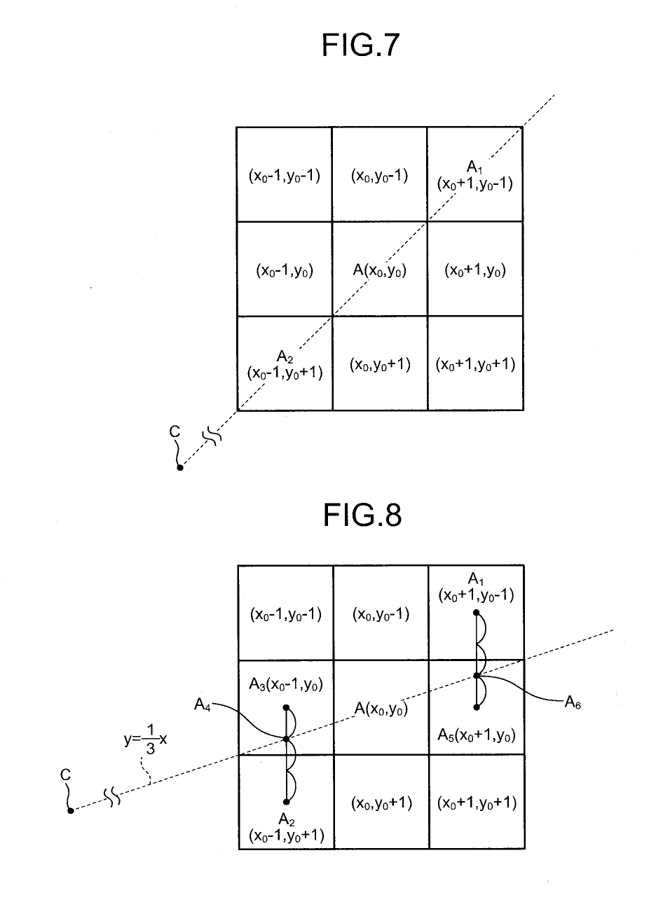

Now, a method for calculating the depth gradient by the depth gradient calculation unit 333 will be described in detail. FIGS. 7 and 8 are schematic diagrams for illustrating a method for calculating the gradient image. Rectangular regions illustrated in FIGS. 7 and 8 indicate the pixel of interest A (x₀, y₀) and its peripheral pixels within the depth image M.

The depth gradient of the pixel of interest A is basically calculated using a pixel value (depth) of the pixel adjacent to the pixel of interest A on a line that connects a center C of the depth image M with the pixel of interest A. For example, as illustrated in FIG. 7, in a case where the centers of pixels A₁ and A₂ adjacent to the pixel of interest A are positioned on the line that connects the center C of the depth image M and the pixel of interest A, a depth gradient G on the pixel of interest A is given by the following formula (4), using vectors CA₁ and CA₂ directing from the center C to the pixels A₁ and A₂, respectively.

$$G = {tan}^{- 1} [ \frac{Z ( {A}_{2} ) - Z ( {A}_{1} )}{\sqrt{{\{ X ( \vec{{CA}_{2}} ) - X ( \vec{{CA}_{1}} ) \}}^{2} + {\{ Y ( \vec{{CA}_{2}} ) - Y ( \vec{{CA}_{1}} ) \}}^{2}}} ] \qquad ( 4 )$$

In formula (4), a sign X( ) represents an x-component of a vector indicated in brackets, and a sign Y( ) represents a y-component of a vector indicated in brackets. Additionally, a sign Z( ) represents a pixel value namely, the depth, of the pixel indicated in brackets.

In contrast, as illustrated in FIG. 8, in a case where the center of pixel adjacent to the pixel of interest A is not positioned on the line that connects the center C of the depth image M and the pixel of interest A, the coordinate and the depth of the adjacent pixel are calculated by linear interpolation using peripheral pixels.

For example, suppose that the center C of the depth image M is the origin, and a line passing through the center C and the pixel of interest A is expressed as y=(1/3)x. In this case, vector CA₄ that gives coordinates of an intersection A₄ of a pixel in a column (x₀−1) and a line y=(1/3)x is calculated by formula (5-1) using vectors CA₂ and CA₃ respectively directed from the center C to pixels A₂ and A₃. Moreover, a depth Z(A₄) at intersection A₄ is given by formula (5-2) using depths Z(A₂), Z(A₃) on the pixels A₂ and A₃.

\vec{CA₄}=⅔\vec{CA₃}+⅓\vec{CA₂} (5-1)

Z(A₄)=⅔Z(A₃)+⅓(A₂) (5-2)

Similarly, vector CA₆ that gives coordinates of an intersection A₆ of the pixel in the row (x₀+1) and the line y=(1/3)x is calculated by formula (6-1) using vectors CA₁ and CA₅, respectively directed from the center C to pixels A₁ and A₅. Moreover, a depth Z(A₆) at the intersection A₆ is given by formula (6-2) using depths Z(A₁), and Z(A₆) on the pixels A₁, and A₅.

\vec{CA₆}=⅔\vec{CA₅}+⅓\vec{CA₁} (6-1)

Z(A₆)=⅔Z(A₅)+⅓(A₁) (6-2)

In this case, the depth gradient G of the pixel of interest A is calculated using the coordinates of the intersections A₄, and A₆ calculated by interpolation and the depth Z(A₄) and Z(A₆) at the intersections A₄ and A₆, similarly to formula (4).

In this manner, the depth gradient calculation unit 333 calculates the depth gradient on all the pixels within the depth image M.

Subsequently, on the basis of the depth gradient calculated by the depth gradient calculation unit 333, the reflected light distribution characteristic calculation unit 334 calculates a value in the reflection angle direction of the distribution characteristics of the illumination light reflected (that is, reflected light) from each point (for example, the point of interest P) on the subject S.

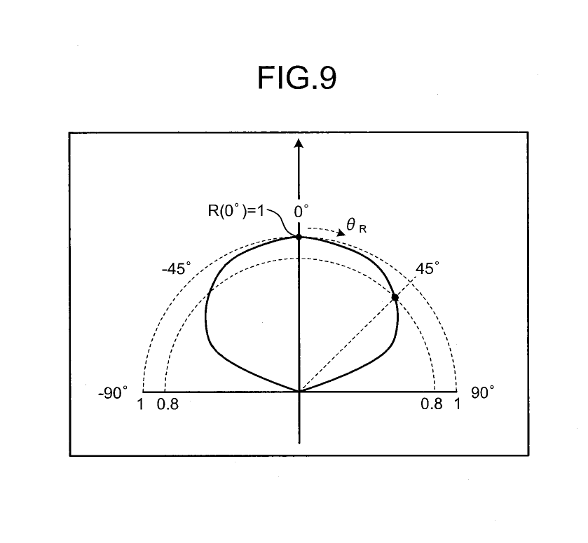

FIG. 9 is a schematic diagram illustrating exemplary light distribution characteristics of the reflected light. The light distribution characteristics of the reflected light refers to a reflectance in accordance with a reflection angle θ_{R} on the surface of the subject S. The light distribution characteristics illustrated in FIG. 9 has been normalized on the basis of reflectance R(θ_{R}=0) when the reflectance is maximum, that is, the reflection angle is θ_{R}=0°. The reflected light distribution characteristic calculation unit 334 reads a function or a table representing the light distribution characteristic illustrated in FIG. 9 from the storage unit 32, calculates the reflection angle θ_{R} from the relationship between the illumination light L1 incident from the illumination unit 21 to the point of interest P, and reflected light L2 emitted from the point of interest P to the collection optical system 22, and then, calculates a value R(θ_{R}) in the reflection angle direction on the light distribution characteristic by applying the function or the table representing the light distribution characteristic.

For example, when the reflection angle θ_{R}=45°, and when the value R(45°) in the reflection angle direction on the light distribution characteristic is such that R(45°)=0.8, the light intensity of the reflected light L2 radiated from the point of interest P in the direction of the image sensor 23 is 0.8 times the light intensity of the case where the reflection angle θ_{R}=0°.

Now, a method for calculating the reflection angle θ_{R} will be described. First, using a technique similar to the case of the illumination light distribution characteristic calculation unit 332, the reflected light distribution characteristic calculation unit 334 calculates a field angle φ, viewed from the pixel A′ on the light-receiving surface 23a, corresponding to the pixel of interest A (refer to FIG. 6) within the depth image M. Moreover, the depth gradient (gradient angle θ) on the pixel of interest A is calculated from the depth gradient calculated by the depth gradient calculation unit 333. Then, the reflection angle θ_{R} is calculated from the field angle θ and a depth gradient (gradient angle θ).

Referring back to FIG. 3, the luminance image creation unit 335 creates a luminance image having the luminance of the subject S image as a pixel value on the basis of the input image data. As illustrated in FIG. 2, since the ranging pixels 23c are sparsely arranged on the light-receiving surface 23a of the image sensor 23, image data has not been obtained at a pixel position in which one of the ranging pixels 23c is disposed. Accordingly, the luminance image creation unit 335 calculates, by interpolation, the luminance at the position of the ranging pixel 23c using image data based on the output value from the imaging pixel 23b located in the vicinity of the ranging pixel 23c.

Subsequently, the image plane illuminance calculation unit 336 calculates illuminance (image plane illuminance) on the image plane E_{f}[lx] of the collection optical system 22 on the basis of the luminance image created by the luminance image creation unit 335. The image plane illuminance herein refers to the illuminance at the time when the reflected light L2 that has passed through the collection optical system 22 is incident into the image sensor 23 when the collection optical system 22 is considered to be an illumination system.

Image plane illuminance E_{f} is given by the following formula (7) using an output value Vₒᵤₜ from the imaging pixel 23b (refer to FIG. 2) of the image sensor 23, a coefficient K, and exposure time t. The coefficient K is an ultimate coefficient that takes into account an absorption coefficient of the light on the imaging pixel 23b, a transform coefficient from electric charge to voltage, and the gain, loss, or the like, on a circuit including those for AD conversion and an amplifier. The coefficient K is predetermined by specifications of the image sensor 23. The image plane illuminance E_{f} at a position of each of the ranging pixels 23c is calculated by interpolation using an output value Vₒᵤₜ from the imaging pixel 23b in the vicinity of the ranging pixel 23c.

$${E}_{f} = {V}_{out} \times \frac{1}{K} \times \frac{1}{t} \qquad ( 7 )$$

Subsequently, the object surface luminance calculation unit 337 calculates object surface luminance L_{S} [cd/m²] that is the luminance on the surface of the subject S on the basis of the image plane illuminance E_{f}. The object surface luminance L_{S} is given by the following formula (8) using the image plane illuminance E_{f}, diameter D of the collection optical system 22, focal length b, and intensity transmittance T(h).

$${L}_{S} = {E}_{f} \times \frac{4}{π} \times \frac{{b}^{2}}{{D}^{2}} \times \frac{1}{T ( h )} \qquad ( 8 )$$

Subsequently, the irradiation illuminance calculation unit 338 calculates irradiation illuminance E₀[lx] of the illumination light L1 emitted on the subject S, on the basis of the object surface luminance L_{S}. By being reflected from the point of interest P of the subject S, the illumination light L1 is attenuated by the reflectance R₀ on the surface of the subject S, while being attenuated by the light distribution characteristic in accordance with the reflection angle θ_{R}. Accordingly, the irradiation illuminance E₀ can be obtained by backward calculation by the following formula (9) using the object surface luminance L_{S}, the reflectance R₀ of the subject S, and the value R(θ_{R}) in the reflection angle direction of the distribution characteristics of the reflected light L2 calculated by the reflected light distribution characteristic calculation unit 334.

$${E}_{0} = {L}_{S} \times \frac{π}{{R}_{0} \cdot R ( {θ}_{R} )} \qquad ( 9 )$$

The reflectance R₀ is a value determined in accordance with the surface property of the subject S and stored in the storage unit 32 beforehand. The storage unit 32 may store a plurality of values of the reflectance R₀ corresponding to the types of subject to be observed such as gastric and colonic mucosa. In this case, the irradiation illuminance calculation unit 338 uses the reflectance R₀ that is selected in accordance with the signal input from the operation input unit 35 (refer to FIG. 1).

The irradiation illuminance E₀ calculated in this manner represents the illuminance level obtained by the process in which the illumination light L1 emitted from the illumination unit 21 reached the point of interest P of the subject S. During this process, the illumination light L1 emitted from the illumination unit 21 is attenuated by the value α(θ_{E}) in the radiation angle direction on the light distribution characteristic in accordance with an irradiation distance d_{L} to the point of interest P, and the radiation angle θ_{E}. Accordingly, the relationship of the following formula (10) is established between luminance L_{LED} of the illumination unit 21 and the irradiation illuminance E₀ on the point of interest P.

$${E}_{0} = \frac{4 \cdot α ( {θ}_{E} ) \cdot {L}_{LED} \cdot {S}_{LED} \cdot E {m}_{SPE}}{{d}_{L}^{2}} \qquad ( 10 )$$

In formula (10), a sign S_{LED} represents a surface area of a region onto which the illumination light L1 is emitted from the illumination unit 21. Moreover, a sign Em_{SPE} represents a spectral characteristic coefficient of the illumination light L1.

Then, the irradiation distance calculation unit 339 obtains, from the illumination light distribution characteristic calculation unit 332, the value α(θ_{E}) in the radiation angle direction of the distribution characteristics of the illumination light, and calculates the irradiation distance d_{L}[m] given by the following formula (11) using the value α(θ_{E}) in the radiation angle direction on the light distribution characteristic, and the irradiation illuminance E₀.

$${d}_{L} = \sqrt{\frac{4 \cdot α ( {θ}_{E} ) \cdot {L}_{LED} \cdot {S}_{LED} \cdot E {m}_{SPE}}{{E}_{0}}} \qquad ( 11 )$$

Subsequently, the subject distance calculation unit 340 calculates a subject distance d_{S}[m] obtained by projecting the irradiation distance d_{L} onto the optical axis Z_{L} by the following formula (12) using the radiation angle θ_{E}.

d_{S}=d_{L}·cos θ_{E} (12)

The depth calculation unit 33b executes the above-described sequential processing on each of the pixels within the depth image M, creates a distance map that associates the calculated subject distance d_{S} to each of the pixels within an image for display, created by the image processing unit 33a, and then, stores the distance map in the storage unit 32. These processes complete processing onto the image data and ranging data obtained from the imaging unit 2.

As described above, according to the first embodiment, a depth image is created on the basis of the ranging data measured by the ranging pixel 23c while the depth gradient is calculated, a value in the radiation angle direction of the distribution characteristics of the illumination light and a value in the reflection angle direction of the distribution characteristics of the reflected light are individually calculated on the basis of the calculated depth image and the depth gradient, and a subject distance is calculated from the luminance of the image using these light distribution characteristic values. Accordingly, it is possible to drastically enhance the accuracy of the subject distance compared with the case where the light distribution characteristic value is not used.

Moreover, according to the first embodiment, the ranging data are obtained from the ranging pixels 23c sparsely arranged on the light-receiving surface 23a of the image sensor 23. Accordingly, it is possible to drastically reduce the data processing amount on the image sensor 23 and data communication amount from the imaging unit 2 to the image processing apparatus 3. Accordingly, this makes it possible to suppress reduction of the imaging frame rate on the image sensor 23.

Modification

In the above-described first embodiment, the image plane phase difference detection AF sensor in which the plurality of imaging pixels 23b and the plurality of ranging pixels 23c are arranged on the same light-receiving surface 23a, is employed as the image sensor 23. However, the configuration of the image sensor 23 is not limited to this. For example, a typical imaging element, such as a CMOS and CCD, may be used in combination with a TOF-system ranging sensor.

### Second Embodiment

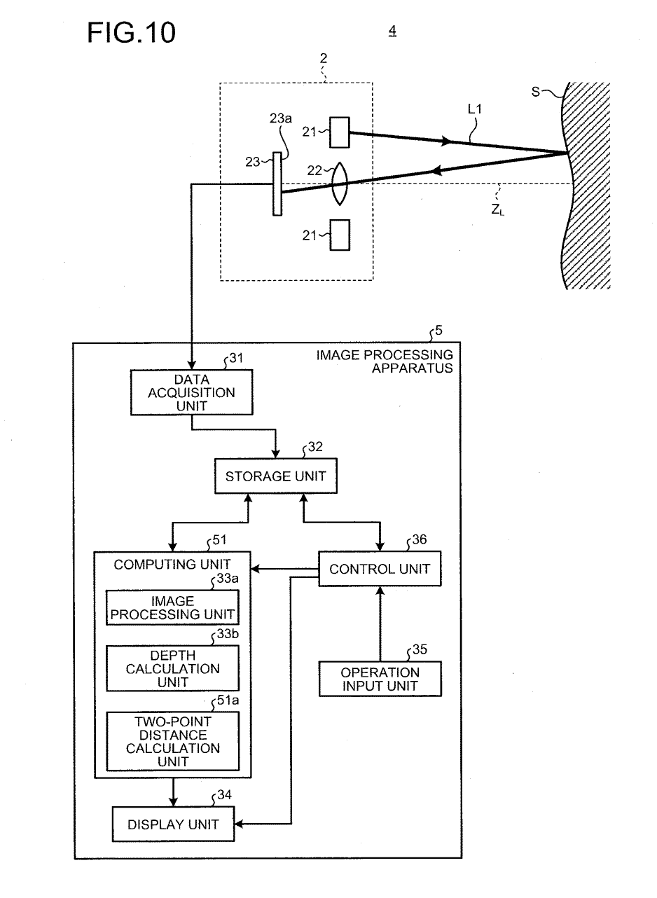

Next, a second embodiment of the present invention will be described. FIG. 10 is a block diagram illustrating a configuration of a ranging system according to a second embodiment of the present invention. As illustrated in FIG. 10, a ranging system 4 according to the second embodiment includes an image processing apparatus 5 instead of the image processing apparatus 3 illustrated in FIG. 1. The configuration and operation of the imaging unit 2 is similar to the case of the first embodiment.

The image processing apparatus 5 includes, instead of the computing unit 33 illustrated in FIG. 1, a computing unit 51 further including a two-point distance calculation unit 51a. The configuration and operation of each element of the image processing apparatus 5 other than the computing unit 51, and operation of the image processing unit 33a and the depth calculation unit 33b included in the computing unit 51, are similar to those of the first embodiment.

On an image for display of the subject S created by the image processing unit 33a, the two-point distance calculation unit 51a calculates a distance between two points designated by a signal input from the operation input unit 35.

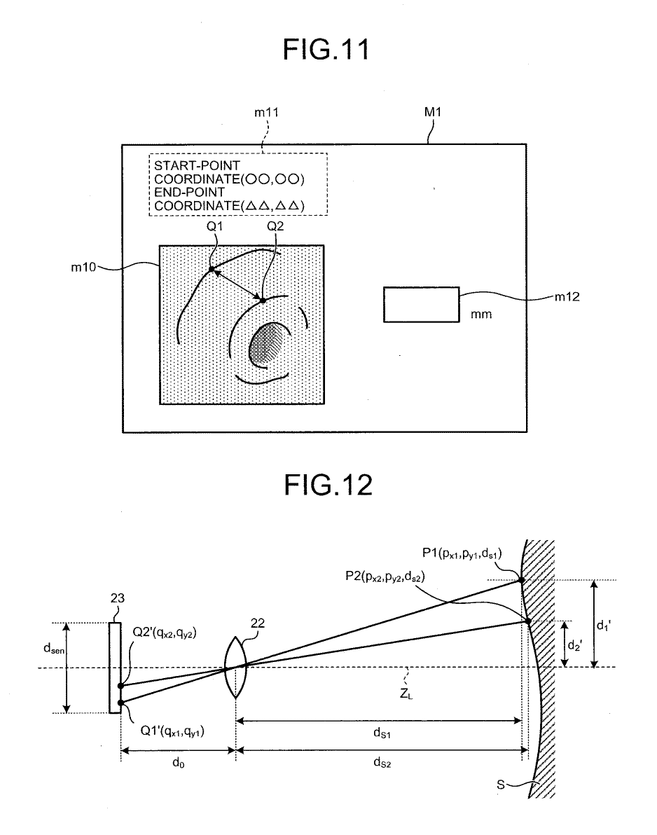

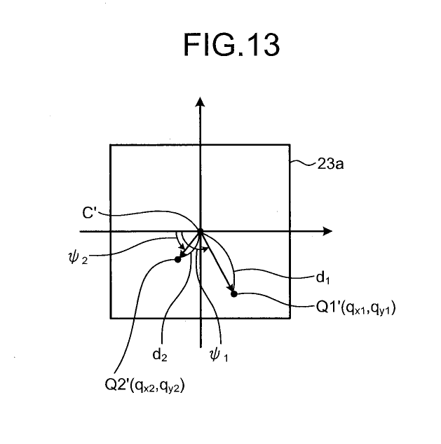

Next, a method for measuring the distance on the subject S, corresponding to a distance between the two points within the image will be described with reference to FIGS. 11 to 13. FIG. 11 is a schematic diagram illustrating an exemplary screen displayed on the display unit 34. FIGS. 12 and 13 are schematic diagrams for illustrating a principle of measuring the distance between two points. Hereinafter, a distance map related to the subject S (refer to the first embodiment) has been created and stored in the storage unit 32.

First, as illustrated in FIG. 11, the control unit 36 displays, onto the display unit 34, a screen M1 including an image m10 for display of the subject S created by the image processing unit 33a. The screen M1 includes, in addition to the image m10, a coordinate display field m11 that displays coordinates of any two points (start point and end point) selected on the image m10 by a user, and includes a distance display field m12 that displays a distance between the two points on the subject S corresponding to any two points selected on the image m10 by the user.

When any two points Q1 and Q2 on the image m10 are designated by predetermined pointer operation (e.g. click operation) onto the screen M1 using the operation input unit 35, the operation input unit 35 inputs coordinate values of the two designated points Q1 and Q2 on the image m10 into the control unit 36.

As described above, the distance map related to the subject S has already been obtained, and therefore, the distance from a point on the subject S corresponding to each of the pixel positions on the image m10, to the imaging unit 2, is known. Moreover, as illustrated in FIG. 12, a sensor size dₛₑₙ and a distance d₀ from the collection optical system 22 to the light-receiving surface 23a are given as design values.

Accordingly, the two-point distance calculation unit 51a obtains coordinate values of the two points Q1 and Q2 on the image m10 from the control unit 36, reads the distance map from the storage unit 32, and obtains distances dₛ₁ and dₛ₂ from two points P1 and P2 on the subject S, corresponding to these two points Q1 and Q2 to the imaging unit 2 (collection optical system 22).

Moreover, as illustrated in FIG. 13, the two-point distance calculation unit 51a obtains coordinate values (qₓ₁, q_{y1}) and (qₓ₂, q_{y2}) of two points Q1′ and Q2′ on the light-receiving surface 23a of the image sensor 23, corresponding to two points Q1 and Q2 on the image m10, and then, calculates image heights d₁ and d₂ (distance from the optical axis Z_{L}) using the obtained coordinate values, the sensor size dₛₑₙ, and the distance d₀. The coordinate values (qₓ₁, q_{y1}) and (qₓ₂, q_{y2}) are coordinates when a point C′ on the light-receiving surface 23a, on which the optical axis Z_{L} passes through, is defined as the origin.

Furthermore, the two-point distance calculation unit 51a obtains rotation angles ψ₁ and ψ₂ from predetermined axes for the vectors respectively directing from the point C′ to the points Q1′ and Q2′.

Subsequently, the two-point distance calculation unit 51a calculates heights d₁′ and d₂′ of the subject (distance from the optical axis Z_{L}) at the points P1 and P2, respectively, on the basis of the image heights d₁ and d₂, the distance d₀ from the collection optical system 22 to the light-receiving surface 23a, and the distances dₛ₁ and dₛ₂ from the points P1 and P2 on the subject S to the collection optical system 22.

When the rotation angles ψ₁ and ψ₂ and the heights of the subject d₁′ and d₂′ illustrated in FIG. 13 are used, the coordinates (pₓ₁, p_{y1}, d_{S1}) and (pₓ₂, p_{y2}, d_{S2}) of the points P1 and P2 on the subject S are respectively given by the following formulas (13) and (14).

(pₓ₁, p_{y1}, d_{S1})=(d₁′ cos ψ₁, d₁′ sin ψ₁, d_{S1}) (13)

(pₓ₂, p_{y2}, d_{S2})=(d₂′ cos ψ₂, d₂′ sin ψ₂, d_{S2}) (14)

The two-point distance calculation unit 51a calculates a distance d between these coordinates (pₓ₁, p_{y1}, d_{S1}) and (pₓ₂, p_{y2}, d_{S2}), outputs the result to the display unit 34 to be displayed, for example, in a distance display field m12 of the screen M1. The distance d may be a distance on a surface orthogonal to the optical axis Z_{L} calculated from two-dimensional coordinates (pₓ₁, p_{y1}) and (pₓ₂, p_{y2}), or may be a distance in a three-dimensional space, calculated from three-dimensional coordinates (pₓ₁, p_{y1}, d_{S1}) and (pₓ₂, p_{y2}, d_{S2}).

As described above, according to the second embodiment of the present invention, it is possible to accurately calculate the distance between the two points on the subject S, corresponding to any two points designated on the image m10, by using the distance map associated with each of the pixels within the image m10.

### Third Embodiment

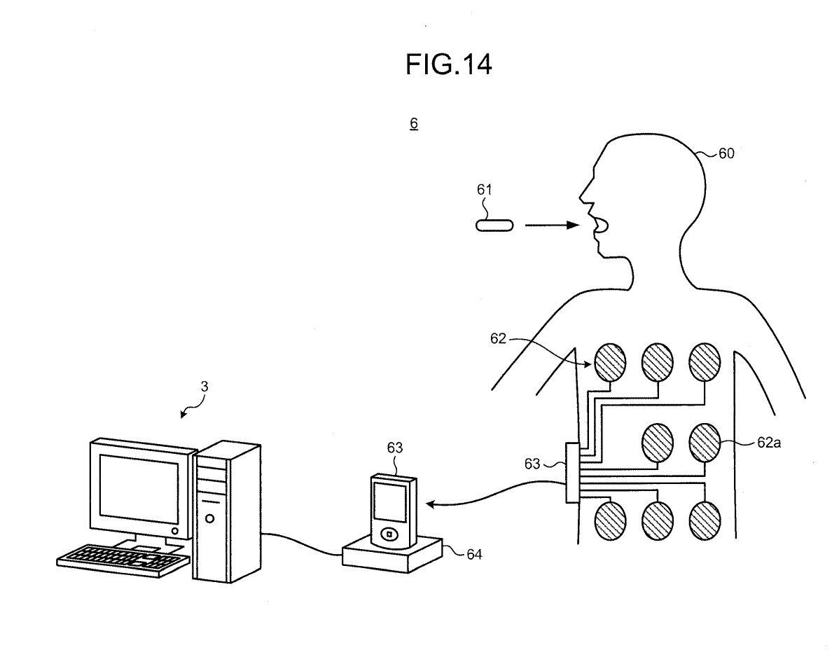

Next, a third embodiment of the present invention will be described. FIG. 14 is a schematic diagram illustrating a configuration of an endoscope system according to the third embodiment of the present invention. As illustrated in FIG. 14, an endoscope system 6 according to the third embodiment includes: a capsule endoscope 61 that is introduced into a subject 60, performs imaging, generates an image signal, and wireless transmits the image signal; a receiving device 63 that receives the image signal wirelessly transmitted from the capsule endoscope 61 via a receiving antenna unit 62 attached on the subject 60; and the image processing apparatus 3. The configuration and operation of the image processing apparatus 3 are similar to the case of the first embodiment (refer to FIG. 1). The image processing apparatus 3 obtains image data from the receiving device 63, performs predetermined image processing on the data, and displays an image within the subject 60. Alternatively, the image processing apparatus 5 according to the second embodiment may be employed instead of the image processing apparatus 3.

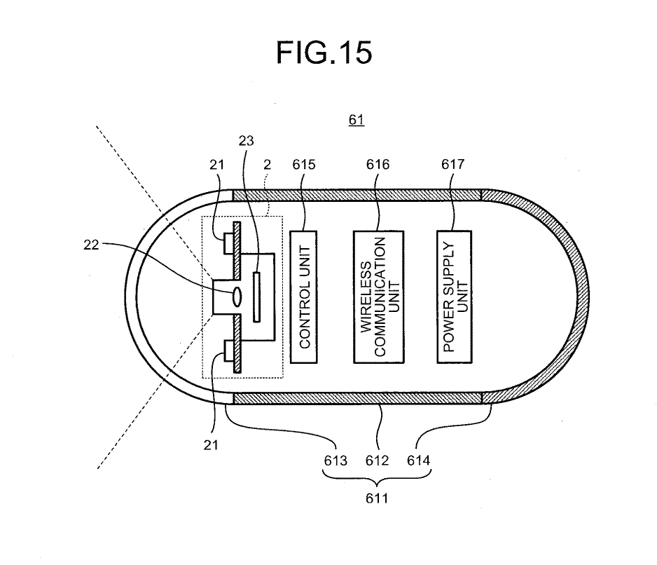

FIG. 15 is a schematic diagram illustrating an exemplary configuration of the capsule endoscope 61. The capsule endoscope 61 is introduced into the subject 60 by oral ingestion, or the like, moves along the gastrointestinal tract, and is finally discharged to the outside from the subject 60. During that period, while moving inside the organ (gastrointestinal tract) by peristaltic motion, the capsule endoscope 61 sequentially generates image signals by imaging inside the subject 60, and wirelessly transmits the image signals.

As illustrated in FIG. 15, the capsule endoscope 61 includes a capsule-shaped casing 611 configured to contain the imaging unit 2 including the illumination unit 21, the collection optical system 22, and the image sensor 23. The capsule-shaped casing 611 is an outer casing formed into a size that can be introduced into the inside of the organ of the subject 60. Moreover, the capsule-shaped casing 611 includes a control unit 615, a wireless communication unit 616, and a power supply unit 617. The control unit 615 controls each element of the capsule endoscope 61. The wireless communication unit 616 wirelessly transmits the signal processed by the control unit 615 to the outside of the capsule endoscope 61. The power supply unit 617 supplies power to each element of the capsule endoscope 61.

The capsule-shaped casing 611 includes a cylindrical casing 612 and dome-shaped casings 613 and 614, being implemented by closing both ends of the openings of the cylindrical casing 612 by the dome-shaped casings 613 and 614. The cylindrical casing 612 and the dome-shaped casing 614 is a casing substantially opaque for the visible light. In contrast, the dome-shaped casing 613 is an optical member having a dome-like shape, transparent to the light having a predetermined wavelength band, such as visible light. The capsule-shaped casing 611 configured in this manner contains, using fluid-tight sealing, the imaging unit 2, the control unit 615, the wireless communication unit 616, and the power supply unit 617.

The control unit 615 controls operation of each element of the capsule endoscope 61 and controls input and output of signals between these elements. Specifically, the control unit 615 controls imaging frame rate of the image sensor 23 of the imaging unit 2, and causes the illumination unit 21 to emit light in synchronization with the imaging frame rate. Moreover, the control unit 615 performs predetermined signal processing on an image signal output from the image sensor 23 and wirelessly transmits the image signal from the wireless communication unit 616.

The wireless communication unit 616 obtains an image signal from the control unit 615, generates a wireless signal by performing modulating processing, or the like, on the image signal, and transmits the processed signal to the receiving device 63.

The power supply unit 617 is a power storage unit such as a button cell battery and a capacitor, and supplies power to each element of the capsule endoscope 61 (imaging unit 2, control unit 615, and wireless communication unit 616).

Referring back to FIG. 14, the receiving antenna unit 62 includes a plurality of (eight in FIG. 14) receiving antennas 62a. Each of the receiving antennas 62a is implemented by a loop antenna, for example, and disposed at a predetermined position on an external surface of the subject 60 (for example, position corresponding to individual organs inside the subject 60, that is, passage region of the capsule endoscope 61).

The receiving device 63 receives an image signal wirelessly transmitted from the capsule endoscope 61 via these receiving antennas 62a, performs predetermined processing on the receiving image signal, and stores the image signal and its related information on an internal memory. The receiving device 63 may include a display unit that displays receiving states of the image signal wirelessly transmitted from the capsule endoscope 61, and an input unit including an operation button to operate the receiving device 63. The image signal stored in the receiving device 63 is transmitted to the image processing apparatus 3 by setting the receiving device 63 on a cradle 64 connected to the image processing apparatus 3.

### Fourth Embodiment

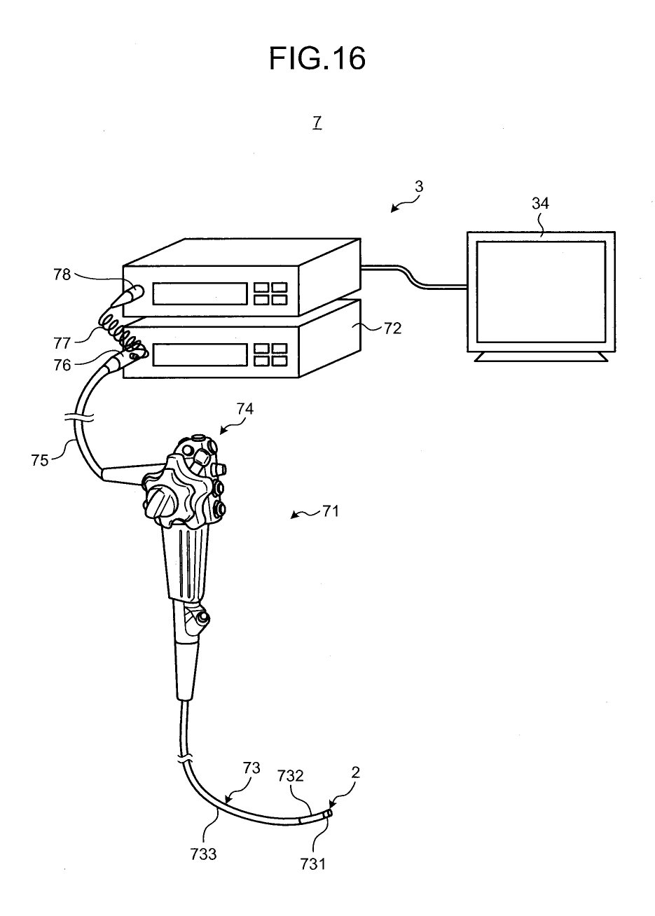

Next, a fourth embodiment of the present invention will be described. FIG. 16 is a schematic diagram illustrating a configuration of an endoscope system according to the fourth embodiment of the present invention. As illustrated in FIG. 16, an endoscope system 7 according to the fourth embodiment includes: an endoscope 71 that is introduced into the body of the subject, performs imaging, creates and outputs an image; a light source apparatus 72 that generates illumination light to be emitted from the distal end of the endoscope 71; and an image processing apparatus 3. The configuration and operation of the image processing apparatus 3 are similar to the case of the first embodiment (refer to FIG. 1). The image processing apparatus 3 obtains image data generated by the endoscope 71, performs predetermined image processing on the data, and displays an image within the subject on the display unit 34. Alternatively, the image processing apparatus 5 according to the second embodiment may be employed instead of the image processing apparatus 3.

The endoscope 71 includes an insertion unit 73 that is a flexible and elongated portion, an operating unit 74 that is connected on a proximal end of the insertion unit 73 and receives input of various operation signals, and a universal cord 75 that extends from the operating unit 74 in a direction opposite to the extending direction of the insertion unit 73, and incorporates various cables for connecting with the image processing apparatus 3 and the light source apparatus 72.

The insertion unit 73 includes a distal end portion 731, a bending portion 732 that is a bendable portion formed with a plurality of bending pieces, and a flexible needle tube 733 that is a long and flexible portion connected with a proximal end of the bending portion 45. At the distal end portion 731 of the insertion unit 73, the imaging unit 2 (refer to FIG. 1) is provided. The imaging unit 2 includes the illumination unit 21 that illuminates the inner portion of the subject by illumination light generated by the light source apparatus 72, the collection optical system 22 that collects illumination light reflected within the subject, and the image sensor 23.

Between the operating unit 74 and the distal end portion 731, a cable assembly and a light guide for transmitting light are connected. The cable assembly includes a plurality of signal lines arranged in a bundle, to be used for transmission and reception of electrical signals with the image processing apparatus 3. The plurality of signal lines includes a signal line for transmitting an image signal output from the image element to the image processing apparatus 3, and a signal line for transmitting a control signal output from the image processing apparatus 3 to the imaging element.

The operating unit 74 includes a bending knob, a treatment tool insertion section, and a plurality of switches. The bending knob is provided for bending the bending portion 732 in up-down directions, and in right-and-left directions. The treatment tool insertion section is provided for inserting treatment tools such as a biological needle, biopsy forceps, a laser knife, and an examination probe. The plurality of switches is used for inputting operating instruction signals into peripheral devices such as the image processing apparatus 3 and the light source apparatus 72.

The universal cord 75 incorporates at least a light guide and a cable assembly. Moreover, the end portion of the side differing from the side linked to the operating unit 74 of the universal cord 75 includes a connector unit 76 that is removably connected with the light source apparatus 72, and includes an electrical connector unit 78 that is electrically connected with the connector unit 76 via a coil cable 77 having a coil shape, and is removably connected with the image processing apparatus 3. The image signal output from the imaging element is input into the image processing apparatus 3 via the coil cable 77 and the electrical connector unit 78.

According to some embodiments, by using parameters of illumination light and reflected light calculated based on ranging data indicating a distance to a subject and using image data indicating an image of the subject, it is possible to calculate, with a high degree of accuracy, a depth between an imaging unit and the subject. With this feature, there is no need to actually measure depths for positions of all pixels constituting the image of the subject, which makes it possible to acquire high-accuracy depth information without drastically increasing data processing amount and data communication amount.

The first to fourth embodiments of the present invention have been described hereinabove merely as examples for implementation of the present invention, and thus, the present invention is not intended to be limited to these embodiments. Furthermore, in the present invention, a plurality of elements disclosed in the above-described first to fourth embodiments may be appropriately combined to form various inventions. The present invention can be modified in various manners in accordance with the specification, or the like, and it is apparent from the description given above that various other embodiments can be implemented within the scope of the present invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus for performing image processing based on image data and ranging data output from an image sensor, the ranging data representing a distance between the image sensor and a subject, the image sensor being configured to receive reflected light of illumination light reflected from the subject and to output the image data and the ranging data, the image processing apparatus comprising:
a processor comprising hardware, wherein the processor is configured to:
calculate a parameter of the illumination light emitted onto a point on the subject, based on the ranging data;
calculate a parameter of the reflected light, based on a gradient of a depth on the point on the subject calculated from the ranging data; and
calculate the distance between the image sensor and the subject in a direction orthogonal to a light-receiving surface of the image sensor, based on the image data, the parameter of the illumination light, and the parameter of the reflected light.

2. The image processing apparatus according to claim 1,
wherein the processor is configured to:
create, based on the ranging data, a depth image in which the depth to the point on the subject corresponding to each of pixel positions of an image created based on the image data is defined as a pixel value of each pixel; and
calculate a value of distribution characteristics of the illumination light in a radiation angle direction, based on the depth image.

3. The image processing apparatus according to claim 2,
wherein the processor is configured to perform interpolation on the depth at a pixel position where the ranging data has not been obtained, among the pixel positions of the image, using the ranging data at a pixel position where the ranging data has been obtained.

4. The image processing apparatus according to claim 2,
wherein the processor is configured to:
calculate the gradient of the depth for each of the pixel positions of the image, based on the depth; and
calculate a value of distribution characteristics of the reflected light in a reflection angle direction, based on the gradient of the depth.

5. The image processing apparatus according to claim 1, further comprising:
a display,
wherein the processor is configured to:
create an image based on the image data; and
calculate a distance between two points on the subject corresponding to any two points designated on the image on the display according to a user operation received by an input device.

6. A capsule endoscope system comprising:
the image processing apparatus according to claim 1; and
a capsule endoscope configured to be introduced into the subject.

7. An endoscope system comprising:
the image processing apparatus according to claim 1; and
an endoscope configured to be inserted into the subject.
